(19) United States
(12) Patent Application Publication
(10) Pub. No.: US 2026/0007808 A1
(43) Pub. Date: Jan. 8, 2026

(54) SILK FIBROIN GEL FOR ADHESION PREVENTION MATERIAL, METHOD FOR PRODUCING SAME, ADHESION PREVENTION MATERIAL, AND ADHESION-PREVENTING METHOD

(71) Applicants: CHARLIE LAB INC., Kumamoto-shi (JP); NATIONAL AGRICULTURE AND FOOD RESEARCH ORGANIZATION, Tsukuba-shi (JP); FUJITA ACADEMY, Toyoake-shi (JP); ATSUMARU HOLDINGS INC., Kumamoto-shi (JP)

(72) Inventors: Yusuke KAMBE, Tsukuba-shi (JP); Yusuke KAWANO, Toyoake-shi (JP); Makoto SASAKI, Kumamoto-shi (JP); Hiroki FUCHIGAMI, Yamaga-shi (JP); Tsunenori KAMEDA, Tsukuba-shi (JP)

(73) Assignees: CHARLIE LAB INC., Kumamoto-shi (JP); NATIONAL AGRICULTURE AND FOOD RESEARCH ORGANIZATION, Tsukuba-shi (JP); FUJITA ACADEMY, Toyoake-shi (JP); ATSUMARU HOLDINGS INC., Kumamoto-shi (JP)

(21) Appl. No.: 19/330,510

(22) Filed: Sep. 16, 2025

Related U.S. Application Data
(63) Continuation of application No. PCT/JP2024/000678, filed on Jan. 12, 2024.

(30) Foreign Application Priority Data
Mar. 17, 2023 (JP) 2023-043384

(51) Int. Cl. A61L 31/04; A61L 31/14; C08L 89/00

(52) U.S. Cl. CPC A61L 31/047; A61L 31/145; C08L 89/00

(57) ABSTRACT

The present invention provides an adhesion prevention material, which has low adhesion and growth promoting properties of fibroblasts and low adsorption to serum proteins. The adhesion prevention material comprising a silk fibroin gel from an aqueous solution of a silk fibroin, wherein the silk fibroin is soluble in water and has a weight-average molecular weight of 150 kDa or less.

### CROSS REFERENCE TO THE RELATED APPLICATIONS

This application is a continuation application, under 35 U.S.C. § 111(a), of international application No. PCT/JP2024/000678 filed Jan. 12, 2024, which claims priority to Japanese Application No. 2023-043384, filed on Mar. 17, 2023, the entire disclosures of all of which are herein incorporated by reference as a part of this application.

### TECHNICAL FIELD

The present invention relates to a silk fibroin gel comprising silk fibroin having a relatively low molecular weight and capable of being used as an adhesion prevention material (adhesion barrier) to be applied internally (inside the body).

### BACKGROUND ART

Recent surgical procedures, such as orthopedic (tendon, etc.) surgery, abdominal surgery, and cardiac surgery, have been remarkably advanced. In addition, due to the recent long-term viability, the number of patients undergoing reoperation, as well as further reoperation has been increasing. However, where the previously performed surgery caused adhesion or the like between the surface of the organ and the surface of the surrounding tissues of the organ, a great amount of time and labor is required for detaching the adhesion. There is also a concern that a new wound may be generated at the time of detachment, resulting in unexpected complications. In order to solve these problems, there is a demand for adhesion prevention materials that can be safely used for a human body or an animal body.

On the other hand, in recent years, silk fibroin has been attracting attention as a biomaterial because of its excellent biocompatibility. For example, Non-Patent Document 1 describes that silk fibroin having a decreased number average molecular weight (Mn) can be obtained by adjusting the period for alkaline hydrolysis of silk fibroin within a range of 10 to 180 minutes, and that when human mesenchymal stem cells were cultured on a hydrogel containing the silk fibroin having the reduced molecular weight, cell exhibited low adhesion to the hydrogel.

In addition, Patent Document 1 discloses a silk fibroin matrix having a PEG-modified surface, i.e., the surface of the matrix is functionalized with polyethylene glycol (PEG), and discloses the altering characteristics regarding protein adsorption, cell adhesion, and proliferation on the surface of the silk fibroin matrix.

In addition, Patent Document 1 describes that in order to prevent adhesion, it is important to suppress adhesion and proliferation of mesenchymal stem cells to an affected area.

### CONVENTIONAL ART DOCUMENTS

[Patent Document 1]U.S. Pat. No. 9,427,499
[Non-Patent Document 1]H. H. Kim et al., “Polymer”, 2016, vol. 90, pp. 26-33

### SUMMARY OF THE INVENTION

With respect to the low molecular weight silk fibroin obtained in Non-Patent Document 1, Non-Patent Document 1 describes that human mesenchymal stem cells have reduced adhesion on a hydrogel containing the silk fibroin. However, from the viewpoint of adhesion prevention material, in fact, the adhesion of human mesenchymal stem cells is not critical issue because the human mesenchymal stem cells are related to cell differentiation. In general, the adhesion prevention material is required to have characteristics such as low adhesion to fibroblasts, low promotion of fibroblast proliferation, and low affinity (adsorption) to serum proteins such as fibrinogen. It is a common general knowledge that cells and proteins have their own adhesion and proliferation promotion properties depending on the types of the cells and the proteins, and therefore, it does not mean that the silk fibroin hydrogel with low molecular weight conversion obtained by Non-Patent Document 1 has low adhesion to fibroblasts and reduces proliferation promotion of fibroblasts, and has low adsorption to serum proteins such as fibrinogen.

Further, although Non-Patent Document 1 shows reduction of Mn of silk fibroin, since the weight-average molecular weight (Mw) of the silk fibroin calculated from PDI and Mn is around 300, there is no technical idea of reducing the Mw in Non-Patent Document 1. Furthermore, as is apparent from Comparative Examples shown later, the molecular weight reduction described in Non-Patent Document 1 was insufficient from the viewpoint of reducing the adhesion and proliferation promoting property of fibroblasts and reducing the adsorption to serum proteins.

In addition, although Patent Document 1 describes that the PEG-modified surface of the silk fibroin matrix inhibits adhesion to and promoting proliferation of fibroblasts and mesenchymal stem cells, it is PEG which contributes to the inhibition of adhesion and proliferation promoting properties in Patent Document 1, and therefore Patent Document 1 fails to find that the silk fibroin structure itself is effective.

Accordingly, an object of the present invention is to provide a silk fibroin material having low adhesion to fibroblasts and low promotion of fibroblast proliferation as well as low adsorption to serum proteins, which is suitable for use as an adhesion prevention material.

As a result of intensive studies to achieve the above purpose, the inventors of the present invention have surprisingly found that by gelling a silk fibroin having a reduced weight average molecular weight (Mw) which is a property-defining attribute, it is possible to produce a medical material that has low adhesion to fibroblasts and low promotion of fibroblast proliferation, as well as has low adsorption property to serum proteins, and is suitable for application to the body. Furthermore, the inventors have found that such silk fibroin gel can reduce adhesion of affected area, and have completed the present invention.

That is, the present invention may comprise aspects described below.

### [Aspect 1]

A silk fibroin gel for an adhesion prevention material, wherein the silk fibroin gel includes a silk fibroin having a weight-average molecular weight of 150 kDa or less (preferably 120 kDa or less, more preferably 100 kDa or less, and even more preferably 80 kDa or less).

### [Aspect 2]

The silk fibroin gel of aspect 1, wherein the silk fibroin has a molecular weight distribution (PDI) of 1 to 10 (preferably 1.5 to 8, more preferably 2 to 6, even more preferably 3 to 5).

### [Aspect 3]

The silk fibroin gel of aspect 1 or 2, wherein the silk fibroin gel has a water retention (mg) of 10 mg or more (preferably 20 mg or more, more preferably 30 mg or more, and even more preferably 40 mg or more) per unit weight (mg).

### [Aspect 4]

An adhesion prevention material comprising the silk fibroin gel of any one of aspects 1 to 3.

### [Aspect 5]

The adhesion prevention material of aspect 4, wherein the adhesion prevention material is applied to at least one selected from the group consisting of an injured area of an organ or tissue due to trauma, a cut surface of an organ or tissue after surgery, and a surrounding tissue surface of an organ after surgery.

### [Aspect 6]

A method of producing a silk fibroin gel for adhesion prevention material, comprising: gelling a silk fibroin having a weight-average molecular weight (Mw) of 150 kDa or less (preferably 120 kDa or less, more preferably 100 kDa or less, even more preferably 80 kDa or less).

### [Aspect 7]

The method for producing the silk fibroin gel for adhesion prevention material of aspect 6, comprising: alkaline-treating a refined silk fibroin, wherein the alkaline treatment is performed at 40° C. or less (preferably 0° C. to 40° C., preferably 5° C. to 40° C., more preferably 10° C. to 35° C.) for a time exceeding 180 minutes (preferably 4 hours or more, more preferably hours or more, and even more preferably 15 hours or more).

### [Aspect 8]

The method for producing the silk fibroin gel for adhesion prevention material of aspect 7, comprising: removing an alkaline component from the alkaline-treated silk fibroin to obtain a dealkalinated silk fibroin, and sterilizing the dealkalinated silk fibroin using an autoclave.

### [Aspect 9]

A method for preventing adhesion using the adhesion prevention material of aspect 4 or 5, comprising: applying the adhesion prevention material to at least one selected from the group consisting of an injured area of an organ or tissue due to trauma, a cut surface of an organ or tissue after surgery, and a surrounding tissue surface of an organ after surgery.

In the present specification, the term “gel” is a semi-solid material, and includes a gel material in which plasticity (fluidity) appears when an external force or heat is applied, as well as a gel material having plasticity (fluidity) even when an external force is not applied. In addition, “gelation” means to form the gel. In the present specification, the term “aqueous solution” is a solution containing water as a main solvent, including, for example, a phosphate-buffered saline.

As used herein, singular form, “a,” “an,” and “the” are intended to include plural forms including “at least one” unless the content clearly states otherwise. As used herein, the terms “and/or,” “at least one,” and “one or more” include any and all combinations of the associated listed items.

It should be noted that any combination of at least two components disclosed in the claims and/or the description and/or drawings is included in the present disclosure. In particular, any combination of two or more of the claims recited in the claims is included in the disclosure.

According to the silk fibroin gel for adhesion prevention material of the present disclosure, it is possible to provide a medical material which has low adhesion to fibroblasts and low promotion of fibroblast proliferation as well as low adsorption to serum proteins, in particular, a medical material which can reduce adhesion of affected area in in vivo evaluation system.

### BRIEF DESCRIPTION OF THE DRAWING

The present disclosure will be more clearly understood from the following description of preferred embodiments with reference to the accompanying drawings. However, the embodiments and drawings are merely for illustration and description and should not be utilized to define the scope of the invention. The scope of the invention is defined by the appended claims. In the accompanying drawings, the same part numbers in the drawings indicate the same parts.

FIG. 1 is a graph showing the serum protein (albumin, fibrinogen) adsorption property of silk fibroin gel samples of Example 1 (LMW SF gel) and Comparative Example 1 (HMW SF gel).

FIG. 2 is a graph showing the adhesion state of fibroblasts at 3 hours after seeding fibroblasts on the silk fibroin gel samples of Example 1 (LMW SF gel) and Comparative Example 1 (HMW SF gel) and a tissue culture polystyrene (TCPS) as a control, respectively.

FIG. 3 is a graph showing the changes over time in the number of fibroblasts up to 7 days after seeding fibroblasts on silk fibroin gel samples of Example 4 (6% LMW SF gel) and Comparative Example 2 (6% HMW SF gel), respectively.

FIG. 4 is a graph showing ankle dorsiflexion angles (Dorsiflexion angles) of rats when rats' Achilles tendons were cut and the silk fibroin gel sample (LMW SF gel) of Example 1, hyaluronic acid (Hyaluronic acid), and none (control) were applied to cut sites, respectively. The right hind limbs (Affected) which had undergone surgery of cut Achilles tendon was compared with the left hind limbs (Healthy), which had not undergone surgery.

FIG. 5 is a photograph showing an operation procedure of cutting Achilles tendon of a rat.

FIG. 6 is a photograph showing a measuring procedure of an angle θ in order to determine an ankle dorsiflexion angle (90°−θ) of a rat.

FIG. 7A is a graph showing adhesion length of ileum of rats when each of the silk fibroin gel sample (LMW SF gel) of Example 1 and the saline solution (Saline) was applied to the rat's ileum on which the abrasion was formed.

FIG. 7B is a graph showing the changes in body weight of rats at the time of transplantation and at the time of evaluation (2 weeks after transplantation) when each of the silk fibroin gel sample (LMW SF gel) of Example 1 and the saline solution (Saline) was applied to the rat's ileum on which the abrasion was formed.

FIG. 8A is a photograph showing the rat's ileum on which the abrasion was formed.

FIG. 8B is a photograph showing the rat's ileum when the adhesion prevention material was applied to the formed abrasion site.

FIG. 9A is a photograph showing the rat's ileum at 2 weeks after applying saline (Saline) to the abrasion site which was formed just before the application.

FIG. 9B is a photograph showing the rat's ileum at 2 weeks after applying the silk fibroin gel sample (LMW SF gel) of Example 1 to the abrasion site which was formed just before the application.

### DETAILED DESCRIPTION

### Silk Fibroin Gel

The silk fibroin gel contains a silk fibroin having a weight-average molecular weight (Mw) of 150 kDa or less. Where the weight average molecular weight falls within this specific range, such a silk fibroin gel can reduce adhesion to fibroblasts, promotion of fibroblast proliferation, and adsorption to serum proteins. The weight-average molecular weight of the silk fibroin contained in the silk fibroin gel may be preferably 120 kDa or less, more preferably 100 kDa or less, and even more preferably 80 kDa or less. The lower limit is not particularly limited, but may be, for example, 10 kDa or more. This silk fibroin gel can be used for the adhesion prevention material.

Silk fibroin gels are hydrogels capable of retaining water in a network structure formed by hydrogen bonding or the like between silk fibroin molecular chains. As used herein, various “molecular weights” refer to molecular weights for silk fibroin molecules in the absence of hydrogen bonding or the like. The weight average molecular weight of the silk fibroin is a value measured by the method described in the following Examples.

Silk fibroin is typically known as a type of fibrous protein, in which molecules are arranged regularly and composed mainly of crystalline portions containing glycine, alanine, and serine, and amorphous portions containing tyrosine and others. The silk fibroin is not particularly limited as long as it has such a configuration, and for example, silk fibroin can be obtained from a silk raw material described later. In addition, the silk fibroin may be chemically modified within a range that does not inhibit the effect of the present disclosure. In the present specification, where simply referred to as “silk fibroin”, the definition thereof also includes chemically modified silk fibroin. However, silk fibroin may be silk fibroin in which film surface of the silk fibroin is not modified with polyethylene glycol (PEG).

From the viewpoint of characteristics of silk fibroin as the adhesion prevention material, it is preferable to include unmodified silk fibroin. In the present specification, the term “unmodified silk fibroin” means a silk fibroin which was not subjected to neither a functional group reaction nor crosslinking couple introduction thereinto as well as which has no chemical modification nor grafting reaction at the side chain of the amino acid residue thereof.

The silk fibroin may have a number-average molecular weight (Mn) of 80 kDa or less, preferably 50 kDa or less, more preferably 30 kDa or less, and even more preferably 20 kDa or less. The lower limit is not particularly limited, but may be, for example, 8 kDa or more. The number-average molecular weight of silk fibroin is a value measured by the method described in the following Examples.

The molecular weight distribution of silk fibroin may be 1 or more and 10 or less, preferably 1.5 or more and 8 or less, more preferably 2 or more and 6 or less, and even more preferably 3 or more and 5 or less. It should be noted that the molecular weight distribution indicates the polydispersity index (PDI) obtained by dividing the value of the weight-average molecular weight (Mw) by the value of the number-average molecular weight (Mn).

Further, the silk fibroin may have a peak-top molecular weight (Mp) of 250 kDa or less, preferably 150 kDa or less, and more preferably 50 kDa or less. The lower limit is not particularly limited, but may be, for example, 20 kDa or more. It should be noted that the peak top molecular weight refers to a molecular weight corresponding to the position of the peak top detected by the chromatogram, and is a value measured by the method described in the following Examples.

The silk fibroin gel may have a thermal decomposition temperature of 250° C. to 300° C., preferably 260° C. to 290° C., and more preferably 270° C. to 280° C. Where the thermal decomposition temperature of the silk fibroin gel falls within the specified range, its crystallinity is relatively low. This results in more amorphous regions, which can retain water, thereby increasing the water retention capacity of the gel. The thermal decomposition temperature is a value measured by the method described in the Examples below.

The silk fibroin gel may have a compressive modulus of 1 to 200 kPa, preferably 3 to 160 kPa, and more preferably 5 to 140 kPa. Where the compressive modulus falls within the above range, such a gel can retain its shape well, making it easy to handle as an adhesion prevention material. The compressive modulus of silk fibroin is a value measured by the method described in the Examples below.

The silk fibroin gel may have a water retention of 10 mg or more, preferably 20 mg or more, more preferably 30 mg or more, and even more preferably 40 mg or more per unit weight (1 mg) of dried silk fibroin. The upper limit is not particularly limited, but may be, for example, 100 mg or less. If the water retention amount is too large, it may become too fluidized to maintain the form of the gel. On the other hand, when the water retention amount is too small, the hardness of the gel is too high, and there is a possibility that it is difficult to closely apply the gel on the cut section of the organ after surgery or along the surface of the surrounding tissue of the organ after surgery. The water retention amount of the silk fibroin gel is a value measured by the method described in the following Examples.

The silk fibroin gel may contain a silk fibroin and a component other than water (for example, an additive such as a colorant), but the content of silk fibroin based on the dry weight of the silk fibroin gel may be 90% or more, preferably 95% or more, more preferably 98% or more, and even more preferably 99.9% or more.

For example, the addition of a colorant to the silk fibroin gel allows medical professionals to easily identify the applied area on the organ surface, which may offer practical advantages.

### Adhesion Prevention Material

The present disclosure includes an adhesion prevention material, wherein the adhesion prevention material contains a silk fibroin gel. As used herein, the term “adhesion prevention material” refers to a biomaterial that is applied to an area where adhesion may occur, such as an injured area of an organ or tissue due to trauma, a cut surface of an organ or tissue after surgery, and a surrounding tissue surface of an organ after surgery for the purpose of suppressing “adhesion”, i.e., a condition where organ or tissue surfaces, which are not supposed to be adhered together, become adhered due to inflammation or similar reactions after surgery.

In addition, in the present specification, the “adhesion prevention material” is applicable for preventing postoperative adhesions between organs or tissues across a wide range of surgical operations, such as orthopedic surgery (tendon, etc.), abdominal surgery, and cardiac surgery. In addition, the present disclosure can be used not only in human surgery but also in the surgery of non-human animals such as pets. For example, non-human animals include non-human mammals, including apes, other primates, mice, rats, hamsters, guinea pigs, horses, cows, pigs, sheep, goats, dogs, cats, rabbits, and the like. For example, adhesion prevention materials are effectively used in preventing postoperative adhesion between tissues during reconstruction such as cut Achilles tendon or ligament and surrounding tissues thereof, or are effectively used in preventing adhesion of the intestine or peritoneum with damaged surfaces.

In the present specification, “adhesion prevention” includes not only completely preventing the occurrence of adhesions but also suppressing the occurrence of adhesions, in other words, suppressing the degree of adhesions to a lesser extent.

As used herein, “trauma” refers to damage to an organ or tissue caused by external (mechanical, physical, chemical) forces.

As used herein, “organ” means an organ in the body that has a unique structure and is an anatomical unit each having a specific function, and includes, for example, brain, heart, esophageal, stomach, bladder, small intestine, large intestine, liver, kidney, pancreas, spleen, uterus, and the like.

The term “tissue” as used herein refers to a unit in which cells or cellular products related to each other participate in organogenesis to form a certain function, and includes skin, muscles, tendons, bones, joints, ligaments, blood vessels, pancreatic islets, cornea, and the like.

As used herein, the term “cut surface” refers to a surface exposed to the body by resection or detachment of a part of an organ or tissue by surgery or the like.

In one aspect, the “adhesion prevention material” may comprise a support necessary to maintain the shape of the gel. In another embodiment, the “adhesion prevention material” may consist essentially of the gel.

In addition, in the present specification, the “adhesion prevention material” may be preferably spread to a cut section of a postoperative organ or tissue, a surrounding tissue surface of a postoperative organ, or the like using a narrow brush, a paint brush, a spray, a flat brush, or the like in a finishing step of a surgical operation such as an open operation, or may be administered to the cut section or the surrounding tissue surface using a syringe, a catheter, or the like.

Alternatively, the adhesion prevention material formed in a sheet shape may be fixed to the cut surface of the organ or tissue after surgery, as well as the surrounding tissue surfaces of the organ after surgery, or the like with a closing means such as a suture or a medical stapler.

### Method for Producing Silk Fibroin Gel

The process for producing a silk fibroin gel includes subjecting a silk fibroin having a weight-average molecular weight of 150 kDa or less to gelation. As the silk fibroin to be subjected to gelation, the silk fibroin described above can be used.

Further, the method for producing the silk fibroin gel may include subjecting a refined silk fibroin to alkaline treatment, and the alkaline treatment may be performed at 40° C. or lower for more than 180 minutes.

As the silk fibroin raw material, it is possible to use silk raw materials containing fibroin and sericin, such as cocoons and raw silks produced by insects (silk-producing insects such as lepidopteran insects such as domestic silkworms, wild silkworms, and Japanese oak silkworm; hymenopteran insects such as wasps and honeybees) and by spiders. By refining the silk raw material, refined silk fibroin can be obtained. Silk fibroin can also be obtained from silk glands. Incidentally, the refining can be performed by a known method, for example, a method of swelling and removing the sericin using an alkaline refining agent such as sodium bicarbonate, sodium silicate, and sodium phosphate, a method of decomposing the sericin with a sericinolytic enzyme, a method of removing the sericin by rotting, and the like.

The refined silk fibroin can have reduced molecular weight by treating it with an alkaline solution. The alkaline treatment can be performed, for example, by using an alkali metal hydroxide such as sodium hydroxide or potassium hydroxide, ammonia, or the like as an alkaline treatment agent. The alkaline treatment is preferably performed in a state in which silk fibroin is dissolved in a solvent. For example, the silk fibroin may be dissolved in a neutral salt solution containing a neutral salt such as lithium bromide or calcium chloride. As the timing of adding the alkaline treatment agent, for example, the alkaline treatment may be performed by dissolving silk fibroin with a mixed aqueous solution together with a neutral salt solution while adding an alkaline treatment agent. Alternatively, an alkaline treatment may be performed by dissolving silk fibroin in a neutral salt solution and then adding an alkaline treatment agent.

By performing the alkaline treatment under relatively mild conditions, the weight average molecular weight can be effectively reduced. The temperature of the alkaline treatment during the treatment may be 40° C. or less, preferably 0° C. to 40° C., preferably 5° C. to 40° C., and more preferably 10° C. to 35° C. The time for the alkaline treatment is more than 180 minutes, preferably 4 hours or more, more preferably 10 hours or more, and even more preferably 15 hours or more. The upper limit of the time for the alkaline treatment is not particularly limited, but may be, for example, 48 hours or less. The alkaline treatment is preferably carried out under stirring.

After the alkaline treatment, the neutral salt may be removed (desalting treatment) or the alkaline treatment agent may be removed (dealkalization treatment) by a known method such as dialysis or ultrafiltration.

Further, from the viewpoint of use as a biomaterial after the alkaline treatment and the dealkalization, it is preferable to purify (remove impurities) or sterilize the dealkalized silk fibroin before the gelation. As for the sterilization treatment, a known method such as filter sterilization or autoclave sterilization can be adopted, but it is preferable to perform autoclave sterilization in view of ensuring the sterilized state.

Silk fibroin gel can be obtained by performing the gelation process on the silk fibroin aqueous solution, particularly preferably after refining and/or sterilization treatment. The concentration of the aqueous silk fibroin solution subjected to the gelation process may be adjusted depending on the form and/or physical properties of the desired gel, for example, the concentration of the aqueous silk fibroin solution may be 1 to 10% (w/v), preferably 2 to 8% (w/v), and more preferably 3 to 6% (w/v).

The gelation treatment can be carried out by various known methods, for example, gelation by keeping pH low by adding citric acid or the like, gelation by standing for a long time at 37° C. to 40° C. temperature conditions, gelation by adding organic solvents such as ethanol, gelation by vortexing, gelation by applying an ultrasonic treatment, or gelation by injecting carbon dioxide gas can be carried out. These gelation treatments may be performed singly or in combination of two or more. After the gelation treatment described above, the treated objects may be subjected to further standing at room temperature (for example, 15 to 30° C.) for a predetermined time (for example, 30 minutes to 6 hours) to complete the gelation treatment.

In addition, the silk fibroin aqueous solution may be gelled depending on a desired form of the adhesion prevention material. For example, prior to gelation, the silk fibroin aqueous solution may be loaded in a syringe, catheter, spray device, or similar applicator to be gelled. Then, the adhesion prevention material comprising the silk fibroin gel can be easily administered to a target site in the human or animal body. Alternatively, by leveling the silk fibroin aqueous solution in a flat container (for example, a Petri dish) having a specific shape subsequently inducing gelation, it is possible to obtain a sheet-like adhesion prevention material (gel sheet) for securing the adhesion prevention material at a specific site within the human or animal body. Here, the gel sheet is a sheet-like substance of a semi-solid material, and can be distinguished from a solid film.

### Examples

Hereinafter, the present disclosure will be described in more detail with reference to Examples and Comparative Examples, but the present disclosure is not limited to these Examples and Comparative Examples in any way. In the following Examples and Comparative Examples, various physical properties were measured by the following method.

### [Molecular Weight of Silk Fibroin]

The silk fibroin aqueous solutions each adjusted to 0.5% (w/v) were used as samples. Each of the samples was measured by gel-filtration chromatography (GFC) with the following instrumentation and conditioning. The weight-average molecular weight (Mw), number-average molecular weight (Mn), molecular weight distribution (PDI), and peak-top molecular weight (Mp) of the silk fibroin were calculated using the molecular weight markers as reference materials contained in the low molecular weight (Low Molecular Weight; LMW) Gel Filtration Calibration Kits and in the high molecular weight (High Molecular Weight; HMW) Gel Filtration Calibration Kits (all manufactured by Cytiva Co., Ltd.).
Device: GFC device “AKTA prime plus” manufactured by Cytiva
Separation columns: “HiLoad 16/600 Superdex 200 pg” manufactured by Cytiva
Mobile phase: 500 mM sodium chloride-containing 20 mM sodium phosphate buffer (pH 7.4)
Flow rate: 1 mL/min.
Temperature: room temperature

### [Thermal Decomposition Temperature (° C.) of Silk Fibroin Gel]

The thermal decomposition temperature (° C.) of each of the silk fibroin gels was measured using a thermogravimetric differential thermal analyzer (TG-DTA; “Thermo plus TG8120” manufactured by Rigaku Co., Ltd.). Specifically, each of the silk fibroin gel samples (41 mg to 48 mg) was encapsulated in an aluminum crucible, and nitrogen was flowed at a flow rate of 200 mL/min. The endothermic peak temperature of the DTA-curve was measured when the temperature was elevated at a rate of 10° C./min starting from 30° C. Each of the three samples prepared individually in the same manner was measured, and the average of these values was calculated as the thermal decomposition temperature.

### [Water Retention of Silk Fibroin Gel]

Into a tube (1.5 mL volume) weighed in advance (the weight at this point is referred to as “tube weight”), was loaded 100 μL of silk fibroin gel, and 1 mL ultrapure water was added therein. The resultant was incubated at room temperature for 3 hours. The incubation was further repeated twice by removing the ultrapure water not retained in the fibroin gel, and adding 1 mL of another ultrapure water before incubation. Finally, the ultrapure water not retained in the silk fibroin gel was removed, and the combined weight of the tube and the silk fibroin gel was weighed (the weight at this point is referred to as “tube+wet gel weight”). After that, the tubes were allowed to stand in a constant-temperature dryer (“KM-600V” manufactured by AZONE Co., Ltd.) set at a temperature of 60° C. and incubated overnight to evaporate the water retained in the silk fibroin gel. The combined weight of the tube and the dried silk fibroin gel was weighed (the weight at this point is referred to as “tube+dry gel weight”). Then, the water retention amount was measured according to the following formula. Each of the three samples prepared separately in the same manner was measured, and the average of these values was calculated as the water retention of the silk fibroin gel.

$$Water retention ( mg / mg ) = [ \{ tube + wet gel weight ( mg ) - tube weight ( mg ) \} - \{ tube + dry gel weight ( mg ) - tube weight ( mg ) \} ] / \{ tube + dry gel weight ( mg ) - tube weight ( mg ) \}$$

### [Compressive Modulus of Silk Fibroin Gel]

A sample silk fibroin gel having a diameter of 10 mm and a height of 5 mm was prepared. The compression modulus of the sample was measured using a uniaxial compression test under the following equipment and measurement conditions. In the same manner as this, each of the four samples prepared separately was measured, and the average of these values was calculated as the compressive modulus of the silk fibroin gel.
Equipment: EZ Test, a small desktop tester manufactured by Shimadzu Corporation
Push Diameter: 8 mm
Compression speed: 1% strain/min
Temperature: room temperature
Strain range for elastic modulus calculation: 0% to 5%

### Example 1

### [Preparation of Silk Fibroin Gel]

A silk fibroin gel according to the example was prepared as follows.

First, a refined silk fibroin (3 g), prepared using an aqueous 0.02 M sodium carbonate solution, was immersed in 50 mL of a mixed aqueous solution containing 9 M lithium bromide and 0.1 M sodium hydroxide (NaOH), and left to stand at room temperature for 1 to 6 hours.

Next, the mixed aqueous solution was stirred at room temperature for 17 hours to dissolve the silk fibroin in the mixed aqueous solution, while an alkaline treatment was performed to obtain a silk fibroin aqueous solution containing a silk fibroin having a relatively low molecular weight.

Subsequently, the obtained silk fibroin aqueous solution was dialyzed (dialysis ratio: 160 times) in deionized water using a dialysis membrane (molecular weight cut-off: 6-8 k). The dialysis procedure was performed at room temperature for 6-12 hours per procedure, which was repeated 6 times. Thus, lithium bromide and sodium hydroxide contained in the silk fibroin aqueous solution were removed.

After removing lithium bromide and sodium hydroxide, the aqueous silk fibroin solution was placed in a dialysis membrane (molecular weight cut-off: 6-8 kDa) and air-dried at room temperature to concentrate the solution. Concentration by air-drying was continued until the volume of the silk fibroin aqueous solution was reduced to ⅕ to ⅓.

The resulting silk fibroin aqueous solution was then subjected to autoclave sterilization process using an autoclave device (“LBS-245” manufactured by Tommy Seiko Co., Ltd.). The autoclave sterilization was performed twice under the same conditions (both at 121° C. for 20 minutes). All subsequent treatments were performed under sterile conditions.

Subsequently, the silk fibroin aqueous solution after autoclave sterilization was centrifuged (40000×g, 20° C., 30 minutes) using a centrifuge device (“Avanti20I” manufactured by Beckman Coulter Co., Ltd.) to remove the precipitated insoluble matter.

After removing the insoluble matter, the silk fibroin aqueous solution was adjusted to have a concentration of 3% (w/v) by appropriately diluting or concentrating the solution, and 1 mL of the solution was collected into a 1.5 mL tube.

Subsequently, the 3% (w/v) silk fibroin aqueous solution was subjected to ultrasonic irradiation (Ampl: 21%, 30 seconds) using an ultrasonic homogenizer (“VCX-750” manufactured by SONICS&MATERIALS).

Then, the tube containing the silk fibroin aqueous solution after the ultrasonic irradiation treatment was spun down to collect the aqueous solution portion from the tube.

The collected silk fibroin aqueous solution was filled in a syringe, and then incubated at room temperature for 4 hours or longer to complete gelation. As a result, the silk fibroin gel according to Example 1 was obtained.

### Example 2

A silk fibroin gel according to Example 2 was obtained in the same manner as in Example 1, except that the concentration of the silk fibroin aqueous solution subjected to ultrasonic irradiation was adjusted to be 4.5% (w/v).

### Example 3

A silk fibroin gel according to Example 3 was obtained in the same manner as in Example 1, except that the concentration of the silk fibroin aqueous solution subjected to ultrasonic irradiation was adjusted to 5% (w/v).

### Example 4

A silk fibroin gel according to Example 4 was obtained in the same manner as in Example 1, except that the concentration of the silk fibroin solution subjected to ultrasonic irradiation was adjusted to 6% (w/v).

### Comparative Example 1

A silk fibroin gel according to Comparative Example 1 was obtained in the same manner as in Example 1, except that the refined silk fibroin (3 g) was immersed in a 9 M lithium bromide aqueous solution (50 mL) that did not contain NaOH, and the duration of the ultrasonic treatment was 10 seconds.

### Comparative Example 2

A silk fibroin gel according to Comparative Example 2 was obtained in the same manner as in Example 1, except that the refined silk fibroin (3 g) was immersed in a 9 M lithium bromide aqueous solution (50 mL) that did not contain NaOH, that the duration of the ultrasonic treatment was set to 10 seconds, and that the concentration of the silk fibroin aqueous solution subjected to the ultrasonic irradiation was adjusted to 6% (w/v).

Table 1 shows the results of various measurements for each of the Examples and Comparative Examples.

TABLE 1

| | | | | | Com. | Com. |
|---|---|---|---|---|---|---|
| | Ex. 1 | Ex. 2 | Ex. 3 | Ex. 4 | Ex. 1 | Ex. 2 |
| Alkaline treatment | Yes | Yes | Yes | Yes | No | No |
| Weight-average | 76.1 | 76.1 | 76.1 | 76.1 | 337.6 | 337.6 |
| molecular | | | | | | |
| weight (Mw) | | | | | | |
| [kDa] | | | | | | |
| Number-average | 17.4 | 17.4 | 17.4 | 17.4 | 44.4 | 44.4 |
| molecular | | | | | | |
| weight (Mn) | | | | | | |
| [kDa] | | | | | | |
| Molecular weight | 4.38 | 4.38 | 4.38 | 4.38 | 7.6 | 7.6 |
| distribution (PDI) | | | | | | |
| Peak-top molecular | 40 | 40 | 40 | 40 | 418 | 418 |
| weight (Mp) | | | | | | |
| [kDa] | | | | | | |
| Thermal | 277 | 280 | 279 | 279 | 285 | 289 |
| decomposition | | | | | | |
| temperature | | | | | | |
| [° C.] | | | | | | |
| Compressive | 11 | 48 | 79 | 121 | 27 | 417 |
| modulus | | | | | | |
| [kPa] | | | | | | |
| Silk fibroin | 3 | 4.5 | 5 | 6 | 3 | 6 |
| concentration | | | | | | |
| [%] | | | | | | |
| Water retention | 51 | 37 | 28 | 21 | 36 | 18 |
| [mg/mg] | | | | | | |

As shown in Table 1, in Examples 1 to 4, where alkaline treatment was performed under specific conditions, the molecular weight of silk fibroin was successfully reduced compared to Comparative Examples 1 and 2. The weight-average molecular weights of silk fibroin in Examples are shown to be 150 kDa or less.

Further, in Examples 1 to 4, since the thermal decomposition temperature can be reduced by lowering the molecular weight, it is considered that the crystallinity of silk fibroin can be reduced. Probably because Examples 1 and 4 had reduced crystallinity and more amorphous regions capable of retaining water, Examples 1 and 4 can improve water retention as compared with Comparative Examples 1 and 2 prepared as corresponding samples with the same concentration, respectively, Further, as shown in Examples 1 to 4, the water retention amount can be adjusted by adjusting the concentration of silk fibroin.

### [Serum Protein Adsorption]

Using a 96-well plate (“Microplate for Tissue Culture (for Adhesive Cells)” manufactured by AGC Technoglass Co., Ltd.) having a well diameter of 7 mm, the silk fibroin aqueous solution after ultrasonic treatment was dispensed into each well and left to stand for gelation as a 100 μL silk fibroin gel sample so that the silk fibroin gel sample formed a horizontal surface inside the well. Specifically, two types of silk fibroin gel samples (LMW SF gel) of Example 1 and silk fibroin gel samples (HMW SF gel) of Comparative Example 1 were prepared in six wells.

Then, onto the silk fibroin gel surface in each well, were added, as 100 μL of serum-protein solutions, either Alexa488-labeled albumin (A13100) manufactured by Invitrogen Co. diluted with phosphate-buffered saline (PBS) manufactured by Gibco Co. to have a 0.45 mg/mL concentration or Alexa488-labeled fibrinogen (F13191) manufactured by Invitrogen Co. diluted with PBS to have a 0.3 mg/mL concentration.

After standing at 37° C. for 1 hour under light-shielded conditions, the serum-protein solutions in the respective wells were removed and the wells were subjected to autoclave sterilization process an autoclave device (“LBS-245” manufactured by Tommy Seiko Co., Ltd.). Thereafter, 200 μL of PBS was added to each well and removed for washing, and such washing treatment was repeated three times. Then, 100 μL of an aqueous SDS solution having a concentration of 10 mg/mL sodium dodecyl sulfate (manufactured by Wako Pure Chemical Industries, Ltd.) was added to each well, and the mixture was allowed to stand at 25° C. for 1 hour under light-shielded conditions, whereby the serum-protein adsorbed on the silk fibroin gel in each well was individually eluted into the aqueous SDS solution. The aqueous SDS solution (50 μL) eluted with serum protein was transferred to each well of another 96-well plate in a one-to-one correspondence, and the adsorption of each serum protein to the silk fibroin gel surface was measured by the following equipment and measuring conditions. The above-described Alexa488-labeled albumin and Alexa488-labeled fibrinogen were used as standards selected from a plurality of serum proteins.
Equipment: Thermo Fisher Scientific microplate reader “Varioskan LUX”
Excitation wavelength: 490 nm
Fluorescent wavelength: 525 nm
Bandwidth: 12 nm
Exposure time: 100 msec

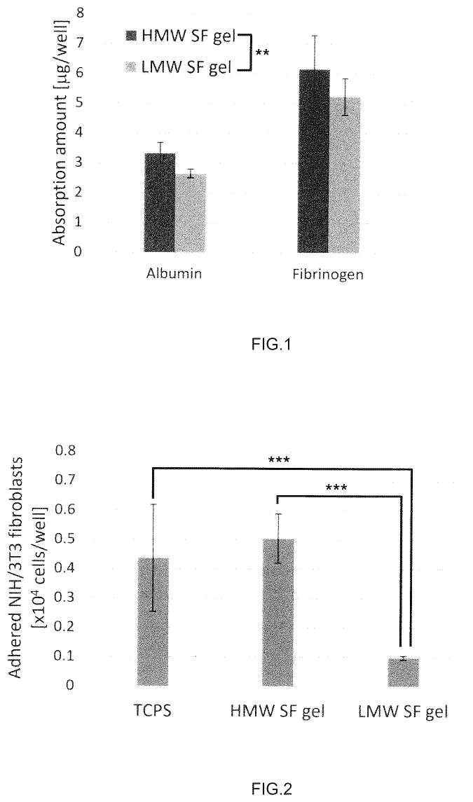

The results obtained thereby are shown in the graph of FIG. 1. FIG. 1 is a graph showing the adsorption to the serum proteins in each of the silk fibroin gel samples of Example 1 (LMW SF gel) and Comparative Example 1 (HMW SF gel). For each of the two serum proteins, the adsorption measurements obtained in 6 wells were averaged, and the average value was adopted as the adsorption value shown in FIG. 1 (n=6). Standard deviations were also determined for each of the six data. It should be noted that the thin vertical lines shown in the respective bar graphs in FIG. 1 represent the mean±standard deviation. In addition, the numbers of asterisks in the Fig show the significance test results (*: p<0.05, **: p<0.01, ***: p<0.001).

As shown in FIG. 1, when albumin as well as fibrinogen were used as the serum proteins, Example 1 (LMW SF gel) has lower serum protein adsorption than Comparative Example 1 (HMW SF gel).

### [Fibroblast Adhesion]

Adhesion of fibroblasts to silk fibroin gel was investigated using murine fetal fibroblasts (NIH/3T3) (sold by JCRB Cell Bank of the National Institute of Health and Nutrition) as cell samples.

The cell samples cryopreserved in a freezer at −80° C. were thawed at 37° C., and suspended in Dulbecco's Modified Eagle's Medium (Gibco) (hereinafter also simply referred to as “medium”) containing 10% fetal bovine serum (purchased from Hyclone) and 1% penicillin-streptomycin (manufactured by Gibco). Then the suspension was plated on a tissue culture dish (manufactured by AGC Technoglass) and incubated in a CO₂ incubator (ACI-165 manufactured by Astec) at 37° C., 5% CO₂, and 100% moisture for 3 days. During the incubation, the medium was changed once after two days passed. The medium was removed, washed with PBS, and then 0.25% tryptic-EDTA solution (manufactured by Gibco) was added to detach the cells from the tissue culture dish.

The solution containing the detached cells was placed in a tube and the medium was added thereto to quench the tryptic-EDTA, and then centrifuged (300×g, room temperature, 3 minutes) to precipitate the cells and remove the supernatant. In the tube, the precipitated cells were washed with PBS (manufactured by Gibco), and then medium was added thereto to suspend the cells. A portion of the cell suspension was collected, and 0.4% trypan blue (manufactured by Gibco) was added to stain the dead cells, and the viable cells were counted using a hemocytometer (manufactured by NanoEntek) to determine the viable cell density in the cell suspension. Thereafter, the cell suspension was diluted by adding the medium, and the viable cell density was adjusted to 1×10⁵ cells/mL.

Using a 96-well plate made of tissue culture polystyrene (TCPS) (a microplate for tissue culture (manufactured by AGC Technoglass Co., Ltd., for adherent cells) having a well diameter of 7 mm), an aqueous silk fibroin solution after ultrasonic treatment was dispensed into each well in order to form a silk fibroin gel sample with a horizontal surface inside the well, and allowed to stand to gelate as a 100 μL silk fibroin gel sample. Two types of silk fibroin gel samples, i.e., Example 1 (low molecular weight, LMW SF gel) and Comparative Example 1 (high molecular weight, HMW SF gel), were each prepared in six individual wells. As a comparative subject, six empty wells (TCPS) were prepared in which the silk fibroin gel sample was not dispensed.

The well plate was allowed to stand at room temperature. The above-described sample of cells (NIH/3T3) was then seeded at 100 μL (1×10⁴ cells) on the gel surface formed inside the wells. The well plate was then allowed to stand in a CO₂ incubator at 37° C., 5% CO₂, and 100% humid for 3 hours. After removing the cell suspension in each well, 200 μL of PBS was added to each well, followed by removing the PBS. Such washing treatment was repeated two times. Then, a TritonX-100 solution having a concentration of 0.5% was prepared by adding PBS to dilute TritonX-100 (manufactured by Sigma-Aldrich), followed by 100 μL of the solution was added to each well. The well plates containing these wells were allowed to stand at 4° C. overnight to lyse the cell membranes of the cell samples. Thus-obtained lysate (10 μL) was used as a sample, and the adhesion of fibroblasts was measured using a cytotoxicity detection-kit PLs (LDH) (manufactured by Roche).

The results obtained thereby are shown in the graph of FIG. 2. FIG. 2 is a graph indicating fibroblast adhesion at the timing of seeding fibroblasts and at 3 hours after seeding thereof onto the silk fibroin gel samples of Example 1 (LMW SF gel) and Comparative Example 1 (HMW SF gel) and tissue culture polystyrene (TCPS) wells as comparative subjects. The measurement results (adsorptive) obtained in 6 wells were averaged, and the average value was adopted as the adhesion value shown in FIG. 2 (n=6). Standard deviations were also determined for each of the six data in FIG. 2. It should be noted that the thin vertical lines shown in each bar graph represent the mean±standard deviation.

As shown in FIG. 2, fibroblast adhesion was lower in Example 1 (LMW SF gel) than in Comparative Example 1 (HMW SF gel).

### [Promotion of Fibroblast Proliferation]

The promotion of fibroblast proliferation to the silk fibroin gel was investigated using mouse fetal fibroblasts (NIH/3T3) (sold by the National Institute of Health and Nutrition JCRB Cell Bank) as cell samples. Cell suspensions were obtained in substantially the same manner as [Fibroblast adhesion] described above, except that the viable cell concentration was adjusted to 4×10⁴ cells/mL by dilution.

Using a 96-well plate (a microplate for tissue culture (manufactured by AGC Technoglass Co., Ltd., for adherent cells) having a well diameter of 7 mm), an aqueous silk fibroin solution (100 μL) after ultrasonic treatment was dispensed into each well in order to form a silk fibroin gel sample with a horizontal surface inside the well, and allowed to stand to gelate to obtain a silk fibroin gel sample. Specifically, two types of silk fibroin gel samples i.e., Example 4 (6% LMW SF gel) and Comparative Example 2 (6% HMW SF gel) were each prepared in four individual wells.

The well plate was allowed to stand at room temperature. The above-described sample of cells (NIH/3T3) was then seeded at 100 μL (4×10³ cells) on the gel surface formed inside the wells. The cells were then cultured in a CO₂ incubator at 37° C., 5% CO₂, and 100% humid for 7 days. During the incubation, the culture medium was changed once after 1 or 2 days. At the timing of initiation of the incubation, and at 1, 3, 5, and 7 after the initiation, 10 μL of WST-1 reagent (“Cell Growth Reagent WST-1” manufactured by Roche Co., Ltd.) was each added to the cell suspension in the respective wells to allow to stand for 1 hour and 30 minutes under conditions of 37° C., 5% CO₂, and 100% moisture.

Subsequently, the cell suspension (100 μL) in each of the wells was transferred to another 96-well plate in a one-to-one correspondence, and the absorbances at the wavelengths 450 nm and 650 nm were measured, respectively, using a microplate reader (“Varioskan LUX” manufactured by Thermo Fisher Scientific). The absorbance at 650 nm was used as a reference absorbance (control), and the difference between the absorbance at 450 nm and the absorbance at 650 nm was calculated as an indication of the number of cells, and the proliferative property of fibroblasts on the silk fibroin gel was measured from the transition.

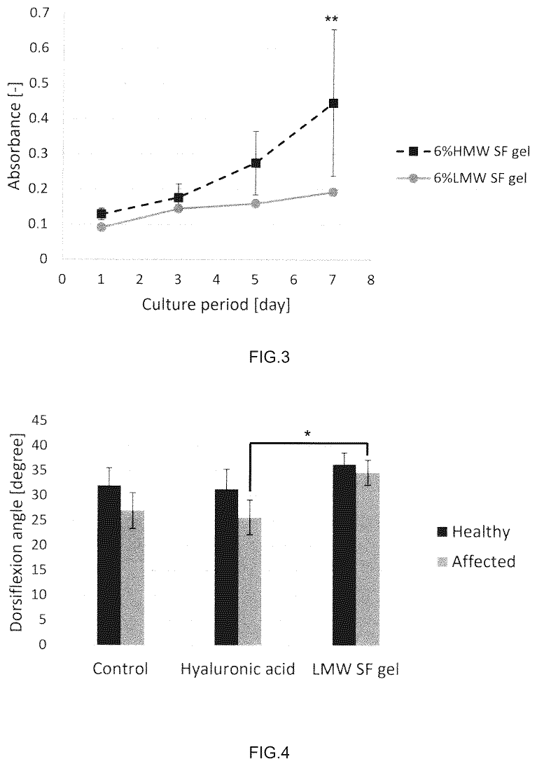

The results obtained thereby are shown in the graph of FIG. 3. FIG. 3 is a graph showing differences in absorbance, i.e., changes in the numbers of fibroblasts over time, measured at a specified wavelength in both samples by day 7 after seeding fibroblasts into silk fibroin gel samples of Example 4 (6% LMW SF gel) and Comparative Example 2 (6% HMW SF gel), respectively. The measurement results of the number of fibroblasts in each day obtained in 4 wells were averaged for each day, and the average value was adopted as the dot value shown in FIG. 3 (n=4). Standard deviations were also determined for each of the four data. It should be noted that the thin vertical lines shown in the dots of the line graph in FIG. 3 represent the mean±standard deviation.

As shown in FIG. 3, in Comparative Example 2 (6% HMW SF gel), the number of fibroblasts was increased with the passage of time, whereas in Example 4 (6% LMW SF gel), the number of fibroblasts was lower than that in Comparative Example 2, indicating suppressed cell proliferative property.

### [Evaluation of Adhesion at Cut Site of Achilles Tendon in Rats]

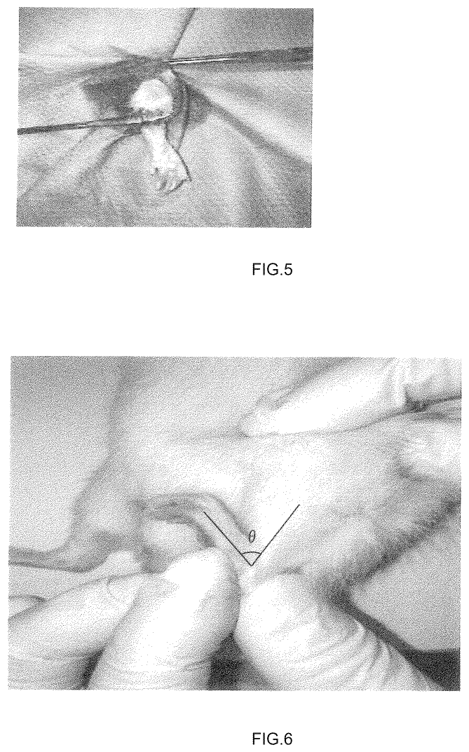

Nine rats (Wistar, 6-week-old male, Nippon SLC) were prepared, and as shown in FIG. 5, for each, an incision was formed in the right hind limb of the rat under general anesthesia to expose the Achilles tendon, and the Achilles tendon was cut in the middle part. The severed Achilles tendon segments were sutured together using a modified Kessler technique with a No. 5-0 nylon suture. Three rats were assigned to each of the three treatment groups, i.e., three rats with no application (Control: control) to the sutured sites of the Achilles tendon, three rats with hyaluronan (Hyaluronic Acid) injection from 1 mL syringes, and three rats with injection of the silk fibroin gel sample (LMW SF gel) of Example 1 from 1 mL syringes. Followed by the incisions in the right hind limb of each rat were closed and allowed to reconstruct autonomously. Four weeks after the operation was performed on the rats, the angle θ shown in FIG. 6 in the left hind limb (healthy side: Healthy) and the right hind limb (treated side: Affected) of each of the rats was measured, and the ankle dorsiflexion angle was calculated as 90°−θ.

Here, the larger the ankle dorsiflexion angle is, the better the ankle bends. In individual rat, the larger the difference in the ankle dorsiflexion angles between the left hind limb and the right hind limb is, the more difficult for bending the ankle is due to adhesion occurring in the right hind limb.

The results obtained thereby are shown in the graph of FIG. 4. FIG. 4 shows a graph of the ankle dorsiflexion angles of rats in each treatment. In each of the rats, the left hind limb (Healthy) had no surgery, while the right hind limb (Affected) underwent surgery. The surgery was performed by cutting the Achilles tendon, followed by applying the cut site in one of three ways: with the low molecular weight silk fibroin gel (LMW SF gel) from Example 1, with hyaluronic acid (Hyaluronic acid), or with no treatment (Control). The graph shows how each treatment affected the ankle dorsiflexion angle. For the ankle dorsiflexion angle, the values of the angles (three values) obtained in three rats prepared under the same conditions were averaged, and this average value was taken as the ankle dorsiflexion angle adopted in FIG. 4 (n=3). Standard deviations were also determined for each of the three data. It should be noted that the thin vertical lines shown in each bar graph represent the mean±standard deviation.

Considering of the evaluation results of adhesion when the adhesion prevention material was applied to the cut site of the Achilles tendon in rats, it is revealed that, as shown in FIG. 4, in the rats (Control) to which nothing was applied, the ankle dorsiflexion angle (27.0±3.6°) of the operated right hind limb was smaller than the ankle dorsiflexion angle (32.0±3.6°) of the non-operated left hind limb, indicating that adhesion occurs between the Achilles tendon and the surrounding tissue after surgery. In the rats (Hyaluronic Acid) to which hyaluronic acid was applied, the ankle dorsiflexion angle (25.7±3.5°) of the operated right hind limb was smaller than the ankle dorsiflexion angle (31.3±4.0°) of the non-operated left hind limb, and it can be seen that the effect of preventing adhesion can hardly be confirmed even when hyaluronic acid, which is known to have effect on smoothing joint movement, was applied.

On the other hand, in the rats (LMW SF gel) to which the adhesion prevention material comprising the silk fibroin gel sample of Example 1 was applied, there was no significant difference between the ankle dorsiflexion angle (34.7±2.5°) of the operated right hind limb and the ankle dorsiflexion angle (36.3±2.3°) of the non-operated left hind limb. Accordingly, it can be seen the significant effect of the LMW SF gel on adhesion prevention.

[Evaluation of Adhesion in Ileums of Rats with Abrasions]

Seven rats (Sprague-Dawley, 7-week-old male, Nippon SLC) were prepared, and each of them was subjected to laparotomy under general anesthesia to expose the ileum of the rat and rubbing a part with 10 cm upstream of the ileocecal portion of the ileum with gauze to form an abrasion. Then, 4 rats were prepared by applying physiological saline (Saline) to the injured part of the ileum, and 3 rats were prepared by injecting the silk fibroin gel sample (LMW SF gel) of Example 1 from syringes. All of the rats were subjected to closing their stomachs. Two weeks after the operation was performed on the rats, the seven rats were again subjected to laparotomy under general anesthesia to remove the ileum, and the length of the portion where the adhesion occurred was measured.

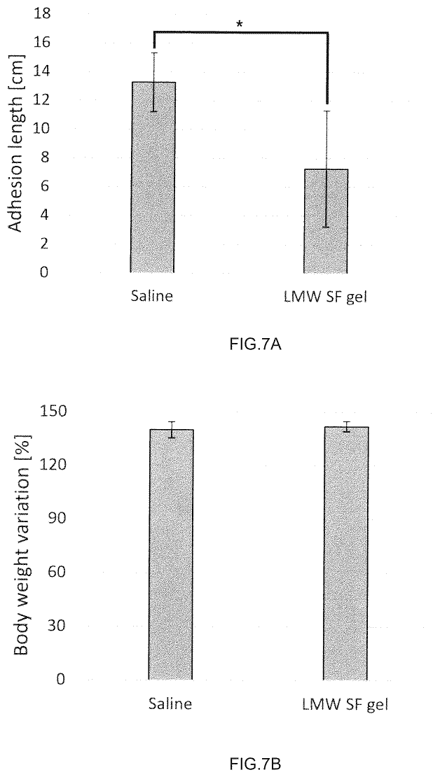

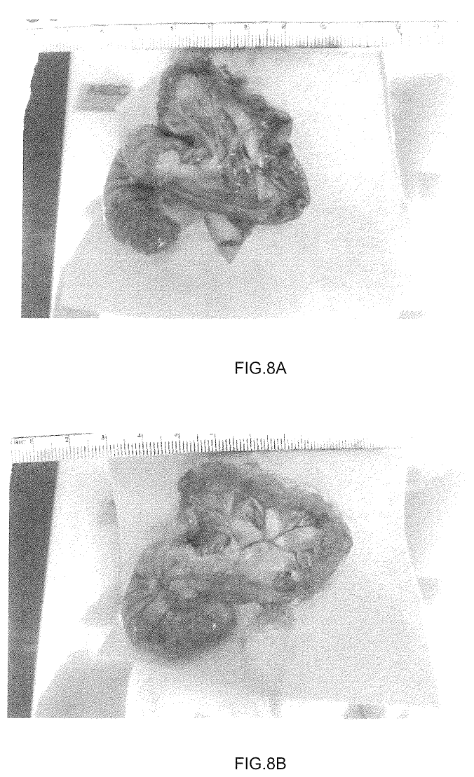

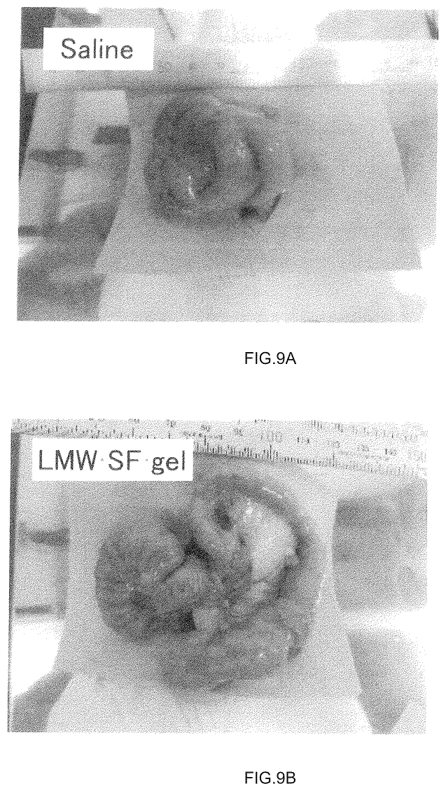

FIG. 7A is a graph showing the adhesion ileum length of the rats subjected to abrasion operation. The abrasion parts of the rats' ileum were treated either with the silk fibroin gel sample (LMW SF gel) of Example 1 or the physiological saline solution (Saline). Regarding the adhesion ileum length, Student t-test was performed for each of the cases in which physiological saline was applied to the injured portion (4 animals) and the cases in which silk fibroin gel samples were applied to the injured portion (3 animals). Significant differences (*: p<0.05) were found between these two groups. It should be noted that the thin vertical lines shown in each bar graph represent the mean±standard deviation.

FIG. 7B is a graph showing the changes in body weight of rats subjected to the ileum abrasion at the time of transplantation and at the time of evaluation (2 weeks after transplantation).

As shown in FIG. 7A, considering the evaluation results by the adhesion prevention material applied to the rat ileum on which the abrasion wound was formed, the adhesion ileum length of the rats in which the silk fibroin gel sample of Example 1 was applied to the injured portion (abrasion-forming portion) was shorter than the adhesion ileum length of the rats in which the saline solution was applied to the injured portion. FIG. 7B shows that there was little difference in body weight changes between the rats treated with silk fibroin gel and those treated with saline on the injured portions. Accordingly, the main difference observed was in the degree of adhesion that formed in the ileum. This reveals that the silk fibroin gel used in Example 1 was more effective at reducing adhesion formation compared to saline.

Based on the results above, it is clear that the silk fibroin gel used in Example 1 showed a remarkable adhesion preventing effect in the in vivo test.

### INDUSTRIAL APPLICABILITY

The silk fibroin gel for adhesion prevention material according to the present disclosure, the method for producing the same, and the method for adhesion prevention can be used in various surgical procedures such as orthopedic surgery (tendon, etc.), abdominal surgery, and cardiac surgery, and can be safely used in the body.

While preferred embodiments of the present disclosure have been described above, those skilled in the art will readily envision various changes and modifications within the obvious scope of the present specification.

Such changes and modifications are, therefore, to be construed as within the scope of the disclosure as defined by the appended claims.

What is claimed is:

1. An adhesion prevention material comprising a silk fibroin gel from an aqueous solution of a silk fibroin, wherein the silk fibroin is soluble in water and has a weight-average molecular weight of 150 kDa or less.

2. The adhesion prevention material of claim 1, wherein the silk fibroin has a molecular weight distribution (PDI) of 1 to 10.

3. The adhesion prevention material of claim 1, wherein the silk fibroin gel has a water retention (mg) of 10 mg or more per unit weight (mg).

4. The adhesion prevention material of claim 4, wherein the silk fibroin gel has a thermal decomposition temperature of 250° C. to 300° C.

5. The adhesion prevention material of claim 1, wherein the silk fibroin gel has a compressive modulus of 1 to 200 kPa.

6. The adhesion prevention material of claim 1, wherein the adhesion prevention material consists essentially of the silk fibroin gel having a silk fibroin content of 90% or more based on the dry weight of the silk fibroin gel.

7. A method of preventing or inhibiting formation of adhesions, comprising:
applying an effective amount of the adhesion prevention material recited in claim 1 to at least one selected from the group consisting of an injured area of an organ or tissue due to trauma, a cut surface of an organ or tissue after surgery, and a surrounding tissue surface of an organ after surgery.

8. The preventing or inhibiting method of claim 7, wherein the silk fibroin has a molecular weight distribution (PDI) of 1 to 10.

9. The preventing or inhibiting method of claim 7, wherein the silk fibroin gel has a water retention (mg) of 10 mg or more per unit weight (mg).

10. The preventing or inhibiting method of claim 9, wherein the silk fibroin gel has a thermal decomposition temperature of 250° C. to 300° C.

11. The preventing or inhibiting method of claim 7, wherein the silk fibroin gel has a compressive modulus of 1 to 200 kPa.

12. The preventing or inhibiting method of claim 7, wherein the adhesion prevention material consists essentially of the silk fibroin gel having a silk fibroin content of 90% or more based on the dry weight of the silk fibroin gel.

13. The preventing or inhibiting method of claim 7, the adhesion prevention material is spread using at least one selected from the group consisting of a narrow brush, a paint brush, a spray, and a flat brush, or is administered using at least one selected from the group consisting of a syringe and a catheter.

14. A method of producing a silk fibroin gel, comprising:
treating an aqueous solution of a silk fibroin having a weight-average molecular weight (Mw) of 150 kDa or less to be gelled.

15. The producing method of claim 14, wherein the silk fibroin has a molecular weight distribution (PDI) of 1 to 10.

16. The producing method of claim 14, wherein the concentration of the aqueous silk fibroin solution is 1 to 10% (w/v).

17. The producing method of claim 14, further comprising:
alkaline-treating a solution of a refined silk fibroin to decrease a weight-average molecular weight (Mw) of the silk fibroin to 150 kDa or less,
wherein the alkaline treatment is performed at 40° C. or less for a time exceeding 180 minutes.

18. The producing method of claim 17, further comprising:
removing an alkaline component from the alkaline-treated silk fibroin solution to obtain a dealkalinated silk fibroin solution, and
sterilizing the dealkalinated silk fibroin solution using an autoclave.
